(19) United States
(12) Patent Application Publication
(10) Pub. No.: US 2026/0334786 A1
(43) Pub. Date: Oct. 29, 2026

(54) SYSTEM AND METHOD FOR MONITORING BIOMECHANICAL CHARACTERISTICS OF AN EYE

(71) Applicant: BAR ILAN UNIVERSITY, Ramat Gan (IL)

(72) Inventors: Zeev ZALEVSKY, Rosh HaAyin (IL); Zeev KALYUZHNER, Holon (IL); Yafim BEIDERMAN, Tel-Aviv (IL); Sergey AGDAROV, Rishon LeTsiyon (IL)

(21) Appl. No.: 19/124,146

(22) PCT Filed: Nov. 1, 2023
(86) PCT No.: PCT/IL2023/051124

§ 371 Date: Apr. 24, 2025

Related U.S. Application Data
(60) Provisional application No. 63/382,875, filed on Nov. 8, 2022.

(51) Int. Cl. A61B 3/16; A61B 3/00

(52) U.S. Cl. CPC A61B 3/165; A61B 3/0008; A61B 3/0025

(57) ABSTRACT

Some embodiments relate to a monitoring system is presented for use in monitoring intraocular pressure. The system comprises a control system configured as a computer system comprising data input and output utilities, memory and a data processor, wherein said data processor is configured and operable to process data indicative of measured data corresponding to sequence of acquisitions indicative of time variation of speckle patterns reflected from a region of interest in individual's eye while being subjected to coherent illumination and cyclic variation of predetermined acoustic stimulation and evaluate an intraocular pressure (IOP) level of the eye, said data processor comprising an IOP classifier configured and operable to apply a first predetermined model-based processing to said data indicative of the measured data and extract measured IOP range of the eye, and generate classification data indicative of the measured IOP range with respect to its relation to a normal IOP range.

### CROSS-REFERENCE TO RELATED APPLICATIONS

This application is a national phase filing under 35 C.F.R. § 371 of and claims priority to PCT Patent Application No. PCT/IL2023/051124, filed on Nov. 1, 2023, which claims the priority benefit under 35 U.S.C. § 119 of U.S. Provisional Patent Application No. 63/382,875, filed on Nov. 8, 2022, the contents of which are hereby incorporated in their entireties by reference.

### TECHNOLOGICAL FIELD

The present disclosure is in the field of medical devices and relates to a system and method for monitoring biomechanical characteristics of an eye. In particular, the presently disclosed subject matter relates to evaluation of intraocular pressure.

### BACKGROUND ART

References considered to be relevant as background to the presently disclosed subject matter are listed below:
1. Wang, W. W., Wang, K.-J., Tsai, C.-L. & Wang, I.-J. Study of noncontact air-puff applanation tonometry IOP measurement on irregularly shaped corneas. in Biomedical Imaging and Sensing Conference vol. 10251 102511X (SPIE, 2017).
2. Zimmermann, M., Pitz, S., Schmidtmann, I., Pfeiffer, N. & Wasielica-Poslednik, J. Tonographic effect of ocular response analyzer in comparison to Goldmann applanation tonometry. PLOS ONE 12, (2017).
3. Koprowski, R. & Wilczyński, S. Corneal Vibrations during Intraocular Pressure Measurement with an Air-Puff Method. Journal of Healthcare Engineering vol. 2018 Preprint at https://doi.org/10.1155/2018/5705749 (2018).
4. Zalevsky, Z. et al. Simultaneous remote extraction of multiple speech sources and heart beats from secondary speckles pattern. Optics Express 17, 21566 (2009).
5. Beiderman, Y. et al. Remote estimation of blood pulse pressure via temporal tracking of reflected secondary speckles pattern. Journal of Biomedical Optics 15, 061707 (2010).
6. Kalyuzhner, Z., Agdarov, S., Bennett, A., Beiderman, Y. & Zalevsky, Z. Remote photonic sensing of blood oxygen saturation via tracking of anomalies in micro-saccades patterns. Optics Express 29, 3386 (2021).
7. Kalyuzhner, Z. et al. Remote photonic detection of human senses using secondary speckle patterns. Scientific Reports 12, (2022).
8. Kalyzhner, Z., Levitas, O., Kalichman, F., Jacobson, R. & Zalevsky, Z. Photonic human identification based on deep learning of back scattered laser speckle patterns. Optics Express 27, 36002 (2019).
9. Bennett, A. et al. Intraocular pressure remote photonic biomonitoring based on temporally encoded external sound wave stimulation. Journal of Biomedical Optics 23, 1 (2018).
10. Camacho-Lopez, S. et al. Intraocular Pressure Study in Ex Vivo Pig Eyes by the Laser-Induced Cavitation Technique: Toward a Non-Contact Intraocular Pressure Sensor. Applied Sciences 10, 2281 (2020).
11. Tan, J., Foster, L. J. R., Lovicu, F. J. & Watson, S. L. Laser-Activated Corneal Adhesive: Retinal Safety in Rabbit Model. Translational Vision Science & Technology 10, 27 (2021).
12. Li, D., Zhang, J., Zhang, Q. & Wei, X. Classification of ECG signals based on 1D convolution neural network. in 2017 IEEE 19th International Conference on e-Health Networking, Applications and Services (Healthcom) 1-6 (IEEE, 2017). doi: 10.1109/HealthCom.2017.8210784.
13. Santurkar, S., Tsipras, D., Ilyas, A. & Madry, A. How Does Batch Normalization Help Optimization? (2018).
14. Agarap, A. F. M. Deep Learning using Rectified Linear Units (ReLU). arXiv 2-8 (2018).
15. Wu, H. & Gu, X. Max-pooling dropout for regularization of convolutional neural networks. Lecture Notes in Computer Science (including subseries Lecture Notes in Artificial Intelligence and Lecture Notes in Bioinformatics) 9489, 46-54 (2015).
16. Li, Z. et al. Teeth category classification via seven-layer deep convolutional neural network with max pooling and global average pooling. International Journal of Imaging Systems and Technology 29, 577-583 (2019).
17. Wang, M., Lu, S., Zhu, D., Lin, J. & Wang, Z. A High-Speed and Low-Complexity Architecture for Softmax Function in Deep Learning. in 2018 IEEE Asia Pacific Conference on Circuits and Systems (APCCAS) 223-226 (IEEE, 2018). doi: 10.1109/APCCAS.2018.8605654.
18. Zhang, Z. & Sabuncu, M. R. Generalized cross entropy loss for training deep neural networks with noisy labels. Advances in Neural Information Processing Systems 2018-Decem, 8778-8788 (2018).

Acknowledgement of the above references herein is not to be inferred as meaning that these are in any way relevant to the patentability of the presently disclosed subject matter.

### BACKGROUND AND BACKGROUND ART

Glaucoma is a major source of irreversible blindness and the second leading cause of the condition, resulting in a global burden on a massive scale, especially in developing countries. The disease is characterized by a series of progressive optic neuropathies, defined by the deterioration of retinal ganglion cells and retinal nerve fiber layers, causing abnormalities in the optical nerve head. High intraocular pressure (IOP) caused by the disease leads to optic nerve degeneration and the death of the retinal ganglion cells.

Glaucoma comes in a variety of forms. Of these, open-angle glaucoma (OAG), normal tension glaucoma, angle-closure glaucoma (ACG), pigmentary glaucoma, and trauma-related glaucoma are the most prevalent. However, the distinction between normal tension glaucoma under normal IOP and ocular hypertension with higher IOP without causing the illness remains unclear.

IOP is a dynamic physiologic factor having regular circadian and random variations over short and extended periods as the subject's muscular tone and physiologic state alternate. Reliable IOP monitoring is thus a critical clinical element in glaucoma management. Although many healthcare glaucoma-related decisions are based on IOP, contemporary glaucoma treatments include frequent IOP assessments during office hours. However, this is a poor resolution that provides inadequate definition of the IOP's fluctuating nature.

Goldmann applanation tonometry (GAT) is the most extensively used ophthalmic tool for measuring IOP. Although GAT is precise, it is influenced by inner-individual variances owing to differences in corneal thickness and stiffness. The method is intrusive and necessitates administration of anesthetic eye drops, limiting IOP monitoring over time. Biochemical features of the cornea affect the accuracy of the applanation tonometry.

The ocular response analyzer (ORA) allows IOP adjustment by considering the biomechanical parameters of the cornea. By directing an ultrasonic wave to the surface of the eye, researchers have been able to evaluate biological pulses, blood flow and mechanical resonance modes of the eye cornea under sound wave stimulation. Despite the fact that such procedures employ sound-driven technology to measure the physical properties of the eyes, no association with IOP has been found. An alternative method of IOP measurement is based on the air puff tonometer, which evaluates IOP based on the resistance of the eye to the air puff. However, over long periods, in order to get full IOP profiles, the method is not suitable.

This above constraint has triggered the need to devise novel ways for continuous IOP monitoring. Several reported examples include implantable telemetric pressure transducers, sensing contact lenses, implantable microfluidic devices, ocular telemetry sensors, and optical devices.

The current methods used for IOP measurement are based on GAT and ORA, and require significant resources, expensive equipment, and close proximity or physical contact with a patient.

Patent publications U.S. Pat. Nos. 9,636,041; 10,398,314; 10,390,729 and WO19111246, all assigned to the assignee of the present application describe optical techniques involving remote evaluation of IOP based on the speckle pattern analyses of detected light response of portion(s) of a cornea surface to applied illumination during a certain time period while under application of external stimulation (pressure field or sound waves stimulation).

Some of these techniques provide for IOP evaluation by analyzing the damping factor of sclera-free oscillations. These techniques for remote laser speckle-based IOP biomonitoring evaluates the intraocular pressure by calculating the damping (Q) factor of transitional oscillations occurring on the surface of eye sclera after terminating its stimulation by a temporally encoded sound wave.

### General Description

There is yet a need in the art for a novel technique for remote (contactless) IOP monitoring with improved precision and enabling to eliminate or at least significantly reduce prior calibration requirements or prior knowledge regarding the IOP level of the eye.

The present disclosure provides a low-cost, remote, photonic IOP biomonitoring. The novel approach of the present disclosure utilizes model-based classification of IOP level data extracted from measured data indicative of time variation of speckle patterns reflected from the eye sclera while being illuminated by coherent light (a laser beam) and subjected to temporally encoded external acoustic (sound) stimulation applied to the eye being illuminated. Depending on the results of this model-based classification, an additional (second) model-based processing of said measured data is selectively performed to determine the IOP value.

The model-based processing utilizes one or more predetermined models, which is/are AI model(s), e.g., Deep Neural Network (DNN), determined using any known in the art AI technique, and such model-based processing performs the classification task according to the principles of the present disclosure.

In the description below, the model-based processing is exemplified as DNN-based technique, but it should be understood that the principles of the presently disclosed subject matter are not limited to the DNN approach.

The above technique can be implemented by a noncontact biomonitoring device which is relatively inexpensive in terms of manufacture and operation. The device can be made compact, is potentially mobile, and is highly accurate even without preliminary calibration or preliminary information on the tested eye.

The inventors succeeded in IOP biomonitoring of 24 eyes of a living creature having characteristics that are closest to the human eye, demonstrating high IOP measurement accuracy and potential clinical utility.

The technique of the present disclosure provides a remote photonic IOP biomonitoring based on temporally encoded external acoustic (sound wave) stimulation, which does not require direct contact with the eye and is inexpensive to build. More specifically, the measurement technique includes projection of coherent light (a laser beam) on eye sclera stimulated by acoustic radiation (a sound wave), and detecting/recording scattered speckle patterns in response to the coherent illumination by a fast-imaging detector (camera).

In order to forecast and effectively reduce background noise from the recorded signal, the inventors created a deep learning driven IOP measurement model. This model allows deep analysis of the recorded signal, considering not only free eye sclera oscillation, but also forced oscillation under periodic stimulation.

The method was successfully tested on 24 pig eyeball samples, given their similarity to the human eye. The tests were conducted by artificial variation of IOP. High detection capacity was achieved without preliminary calibration.

According to one broad aspect of the disclosure, it provides a monitoring system for use in monitoring intraocular pressure, the system comprising a control system being configured as a computer system comprising data input and output utilities, memory and a data processor, wherein the data processor is configured and operable to process data indicative of measured data corresponding to sequence of acquisitions indicative of time variation of speckle patterns reflected from a region of interest in individual's eye while being subjected to coherent illumination and cyclic variation of predetermined acoustic stimulation and evaluate an intraocular pressure (IOP) level of the eye, the data processor comprising an IOP classifier configured and operable to apply a first predetermined model-based processing to said data indicative of the measured data and extract measured IOP range of the eye, and generate classification data indicative of the measured IOP range with respect to its relation to a normal IOP range.

In some embodiments, the data indicative of the measured data comprises one-dimensional vector representation of the measured data.

In some embodiments, the data processor further comprises a pre-processor configured and operable to generate the one-dimensional vector representation of the measured data.

In some other embodiments, the data processor comprises a correlation module configured and operable to calculate cross-correlations between every two consecutive speckle patterns in said sequence of acquisitions and create the one-dimensional vector representation of cross-correlation peaks.

In some embodiments, the predetermined acoustic stimulation comprises excitation frequency in the range of 100 to 500 Hz.

The data processor may further comprise an IOP analyzer configured and operable to analyze the classification data indicative of the measured IOP range and, upon identifying that the measured IOP range is within the normal IOP range, selectively apply a second predetermined model-based processing to said one-dimensional vector representation of the measured data and extract an IOP level of the eye.

The first predetermined model-based processing may comprise utilizing a predetermined generic model and classifying the measured IOP range, based on said one-dimensional vector representation, into one of three predefined classes of IOP ranges being normal, high, and extremely high IOP ranges.

For example, the normal IOP range is 10-21 mm Hg. For example, the high IOP range is 22-33 mm Hg. For example, the extremely high IOP range is 34-45 mm Hg.

The second predetermined model-based processing utilizes individual models and determines the IOP value from the normal IOP range with increasing accuracy level.

In some embodiments, the second predetermined model-based processing utilizes an individual model and determines the IOP value with an accuracy level of 5 mm Hg in the normal IOP range.

In some embodiments, the second predetermined model-based processing utilizes an individual model and determines the IOP value with an accuracy level of 3 mm Hg in the normal IOP range.

In some other embodiments, the second predetermined model-based processing utilizes an individual model and determines the IOP value with an accuracy level of 1 mm Hg in the normal IOP range.

The monitoring system of the disclosure may comprise a first model-based processing utilizing DNN models based on 1D convolutional layers, wherein the first three layers within the DNN model may comprise a combination of 1D convolution, batch normalization and rectified linear unit, followed by a global average pooling operation.

In some embodiments, the control system is configured and operable for data communication with a measured data provider to obtain therefrom said data indicative of the measured data.

The monitoring system may further include a measurement device configured and operable to provide the measured data, the measurement device comprising:
an optical unit configured and operable to perform one or more continuous imaging sessions, the optical unit comprising: a light source configured and operable to provide coherent illumination of at least one selected wavelength to illuminate a region of interest of an eye; and an optical detector detecting light reflected from the region of interest in the eye in response to said illumination and generating measured data for each of the at least one selected wavelength, said measured data comprising a sequence of acquisitions indicative of variation of speckle patterns in said reflected light;
a stimulation unit comprising an acoustic wave generator and applicator, the stimulation unit being configured and operable to perform one or more continuous stimulation sessions on said region of interest while under said imaging session, wherein each continuous stimulation session comprises a predetermined time pattern of stimulation cycles, thus applying to said region of interest a predetermined temporally encoded external acoustic waves, such that said variation of speckle patterns is affected by cyclic variation of the stimulation.

The predetermined temporally encoded external acoustic waves may comprise excitation frequency in a range of 100 to 500 Hz.

According to a further broad aspect of the present disclosure, it provides a monitoring system for monitoring intraocular pressure, the system comprising:
an optical unit configured and operable to perform one or more continuous imaging sessions, the optical unit comprising: a light source configured and operable to provide a coherent illumination of at least one selected wavelength to illuminate a region of interest of an eye; and an optical detector detecting light reflected from the region of interest in the eye in response to said illumination and generating measured data for each of the at least one selected wavelength, said measured data comprising a sequence of acquisitions indicative of variation of speckle patterns in said reflected light;
a stimulation unit comprising a sound wave generator and applicator, the stimulation unit being configured and operable to perform one or more continuous stimulation sessions on said region of interest while under said imaging session, wherein each continuous stimulation session comprises a predetermined time pattern of stimulation cycles, thus applying to said region of interest a predetermined temporally encoded external sound waves, such that said variation of speckle patterns is affected by cyclic variation of the stimulation; and
a control system configured and operable to be responsive to the measured data to process said measured data and evaluate an IOP level of the eye, said processing comprising model-based processing.

In its yet further broad aspect, the present disclosure provides a method for use in monitoring intraocular pressure, the method comprising:
providing data indicative of measured data corresponding to sequence of acquisition indicative of time variation of speckle patterns reflected from illuminated region of interest in individual's eye affected by cyclic variation of predetermined acoustic stimulation;
processing said data indicative of the measured data to evaluate an IOP level of the eye, said processing comprising apply a first predetermined model-based processing to said data and extracting measured IOP range of the eye, and generating classification data indicative of the measured IOP range with respect to its relation to a normal IOP range.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting examples only, with reference to the accompanying drawings, in which:

FIG. 1 is a block diagram of an exemplified monitoring system of the present disclosure for evaluation and measurement of IOP of a subject;

FIG. 2A is a flow diagram of an exemplary method of the present disclosure;

FIG. 2B exemplifies more specifically decision-making procedures performed at the IOP evaluation and IOP personalized measurement stages;

FIGS. 3A and 3B show more specifically an exemplary configuration of the IOP monitoring system of the present disclosure;

FIGS. 4A and 4B show schematically the IOP simulation experimental setup and the pre-processing of the measured speckled images (measured data indicative of scattered speckle patterns being detected);

FIG. 5A exemplifies a confusion matrix of IOP trained generic DNN model, and FIG. 5B shows a 100-millisecond data sample of a pre-processed speckle pattern detected from a single tested eye;

FIGS. 6A to 6C show confusion matrices of three IOP classification tasks performed on a single eye; and

FIGS. 7A to 7C exemplify the DNN model architecture and feature extraction sample, wherein FIG. 7A shows the model architecture; FIG. 7B shows the 64 convolutional layer filters constituting the DNN's last convolutional layer; and FIG. 7C shows that the DNN classified the IOP level either when the eyeball started or when it stopped reacting to the external acoustic stimulation signal.

### DETAILED DESCRIPTION OF EMBODIMENTS

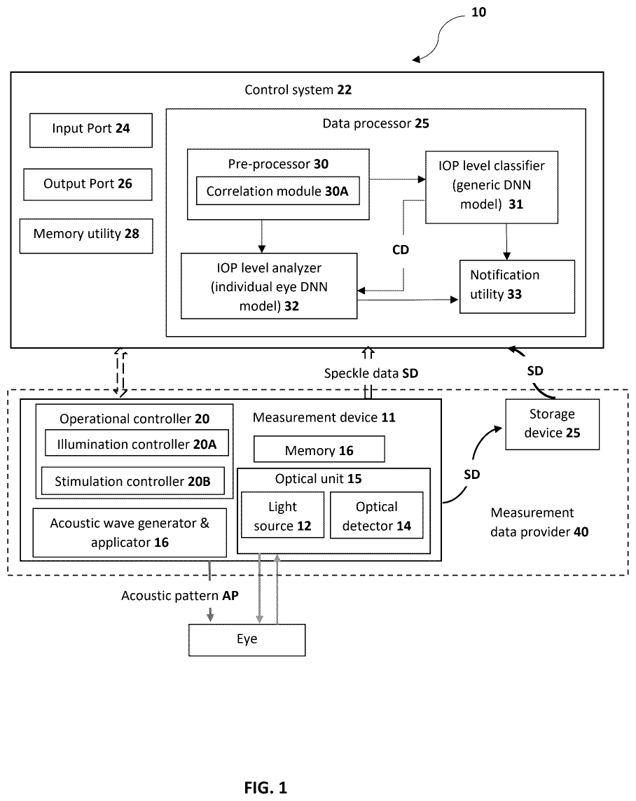

Reference is made to FIG. 1 illustrating, by way of a block diagram, an exemplary monitoring system 10 of the present disclosure for monitoring the subject's intra ocular pressure (IOP). The monitoring system 10 includes a control system 22 is configured and operable to receive and process data indicative of measured data corresponding to time variation of speckle-based image data SD formed by optical response of an eye to coherent illumination over time and concurrent application of a predetermined pattern of acoustic stimulation, to evaluate an IOP level of the eye.

To this end, the control system 22 is configured for data communication with a measured data provider 40 (where the measured data or data indicative of the measured data is properly stored) using wires-based or wireless communication between them of any known suitable type and communication protocols. The control system may be a stand-alone system at a remote station (e.g., network server) being in data communication with a stand-alone storage device 25 presenting the measured data provider and being in communication with a measurement device 11 for receiving therefrom and storing the measured data (at times referred to herein as SD). Alternatively or additionally, the measured data provider may also be associated with internal memory utility 16 of the measurement device 11. According to some other examples, the measurement device 11 and the control system 22 may be integrated in a common system (i.e., functional utilities of the control system 20 are incorporated in a local controller of the measurement device), or functional utilities of the control system 22 may be distributed between the measurement device and the remote station.

The measurement device 11 is configured and operable to perform one or more measurement sessions, each providing acquisition of a sequence of frames (video) indicative of the time variation of speckle-based image data SD formed by optical response of the eye to coherent illumination over time and concurrent application of a predetermined pattern of sound waves stimulation. To this end, the measurement device 11 includes an optical unit 15, and a stimulation unit 16, and may also include an operational controller 20. The stimulation unit 16 is configured as/comprises an acoustic wave generator and applicator unit. The optical unit 15 includes a light source 12 generating coherent light (e.g., laser light) of at least one selected wavelength to illuminate a region of interest of the eye, and an optical detector (camera) 14 which detects the light response (reflection/scattering) of the region of interest to said illumination.

The optical unit 15 is configured to implement defocused imaging of the region of interest in one or more measurement/imaging sessions. The measured data generated by the detector 14 in each measurement session, for each of the at least one selected wavelength, comprises a sequence of acquisitions indicative of speckle-based image data SD, i.e., time variation of speckle patterns in the reflected light.

The measured data is at times referred to herein below as speckle data or speckle-based SD. The acoustic radiation is at times referred to herein below as sound wave(s). The acoustic radiation presents external stimulation field and has a certain acoustic pattern which is at times referred to herein as stimulation sound pattern.

The stimulation unit 16 (acoustic/sound wave generator and applicator) operates to perform one or more continuous stimulation sessions on the region of interest while under the imaging/measurement session (or at least a part thereof). More specifically, the stimulation unit generates sound waves of a predetermined time pattern and applies such acoustic pattern AP, as a stimulation field, to the region of interest during the measurement session. Each continuous stimulation session includes the predetermined time pattern of stimulation cycles, thus applying to the region of interest a predetermined temporally encoded external acoustic radiation (sound waves). Accordingly, the time variation of the speckle patterns being measured is affected by cyclic variation of the stimulation field.

More specifically, the stimulation sound pattern has a certain frequency in a range of 100 to 500 Hz (e.g. 390 Hz) implemented in a sequence of time-spaced pulses (e.g. 1 sec pulses spaced by 1 sec gaps) configured with certain rise and fall segments of the sound pulse. As a result, the speckle-based image data SD being detected (i.e., measured data) is indicative of speckles' variation over time affected by the rises and falls of the pulses. In other words, the speckle-based data is indicative of the speckles' behavior during the rises and falls of the stimulating pulses.

The operational controller 20 may include an illumination controller 20A and/or stimulation controller 20B.

The control system 22 is configured and operable to be responsive to data indicative of the measured data (being received from the measured data provider, i.e., the measurement device and/or an external storage device) to process this measured data (speckle-based image data) and evaluate an IOP level of the eye. The processing utilizes AI-based techniques, e.g., deep neural network (DNN) processing techniques.

Thus, the control system 22 is a computer system including, inter alia, input utility 24, output utility 26, memory utility 28, and data processor 25. The data processor 25 includes an IOP level classifier 31 which is configured and operable to apply to measured data (or data indicative of such measured data) a first predetermined DNN model processing, utilizing a generic DNN model, to extract measured IOP range and generate classification data CD indicative of the measured IOP range with respect to its relation to a normal IOP range (typically 10-21 mm Hg). In other words, the classifier utility operates to classify the measured IOP range between normal and abnormal IOP ranges in a discrete manner. This will be described more specifically further below.

The main goal of IOP level classifier 30 is to identify abnormal IOP levels for further clinical examination. It should be emphasized that such classification-based approach provides for identifying abnormal IOP condition (high and extremely high IOPs) in a relatively simple procedure requiring no calibration or prior knowledge about the tested eye.

The classifier 31 may be in data communication with a notification utility 33 which generates a notification data to an authorized person (e.g. physician) regarding the measured IOP range. For example, such notification is selectively generated in case the measured IOP range is outside the normal range, thus advising an individual that further medical checks are to be conducted.

Preferably, the data processor also includes a pre-processor 30 which performs preliminary processing of the raw measured data (speckle-based image data SD) to generate data indicative of the measured data in the form of one-dimensional vector representation of the measured data. Such pre-processor includes a correlation module 30A which is configured to calculate cross-correlations between every two consecutive speckle patterns in the sequence of acquisitions and create the one-dimensional vector representation of cross-correlation peaks.

The classifier 31 can thus operate to apply said first predetermined DNN model processing to the one-dimensional vector representation of the measured data and generate the classification data CD as described above.

Preferably, the data processor 25 also includes an analyzer 32 which is configured and operable to analyze the classification data CD (measured IOP range) and, depending on whether or not the IOP range is outside the normal range, either operate the notification utility 33 or selectively apply a second predetermined DNN model processing to the measured data (vector representation thereof), utilizing an DNN individual model, to extract the IOP value/level with desired relatively high accuracy level, up to 1 mm Hg. This will be described more specifically further below.

The individual-level DNN processing might require a calibration stage. Calibration is based on trained model which was trained using another known in the art technique of IOP measurement (e.g. Goldman).

As indicated above, the control system 22 may be associated with a separate remote station and include the data processor 25 configured as described above. More specifically, this data processor may be responsive to the raw measured data (speckle data) to determine the one-dimensional vector representation thereof. Alternatively, such preliminary processing may be performed by a local controller of the measurement device 11 and the vector representation is communicated to the remote control system.

Thus, the data processor 25 processes the data indicative of the measured data (i.e., the one-dimensional vector representation) corresponding to time variation of speckle patterns reflected/scattered from illuminated region of interest in individual's eye affected by cyclic variation of predetermined sound waves stimulation and evaluates an IOP level of the eye. As described above, I data processor 25 includes the IOP classifier 31 configured and operable to apply the first predetermined DNN model processing to the one-dimensional vector representation of the measured data and extract the measured IOP range of the eye, and generate the classification data indicative of the measured IOP range with respect to its relation to the normal IOP range.

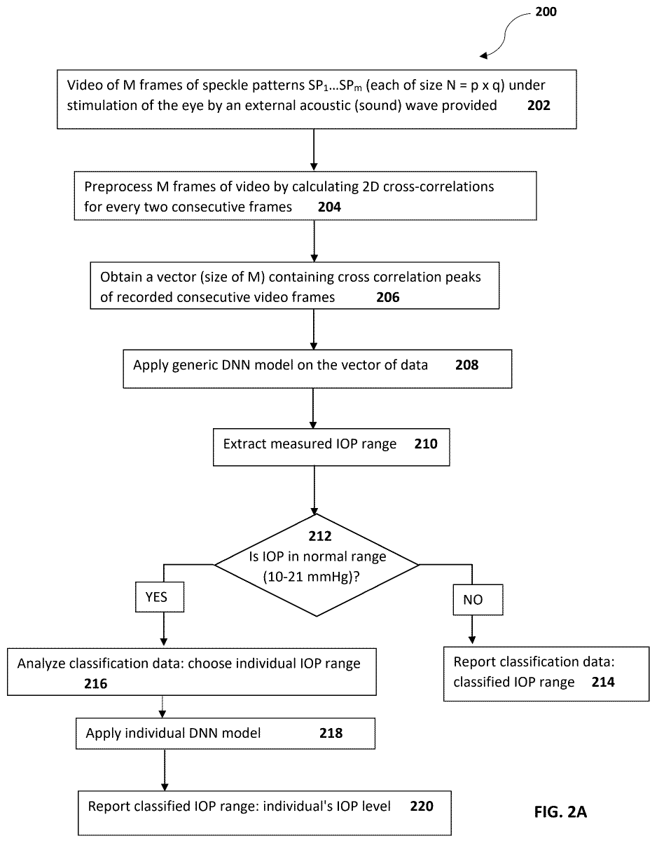

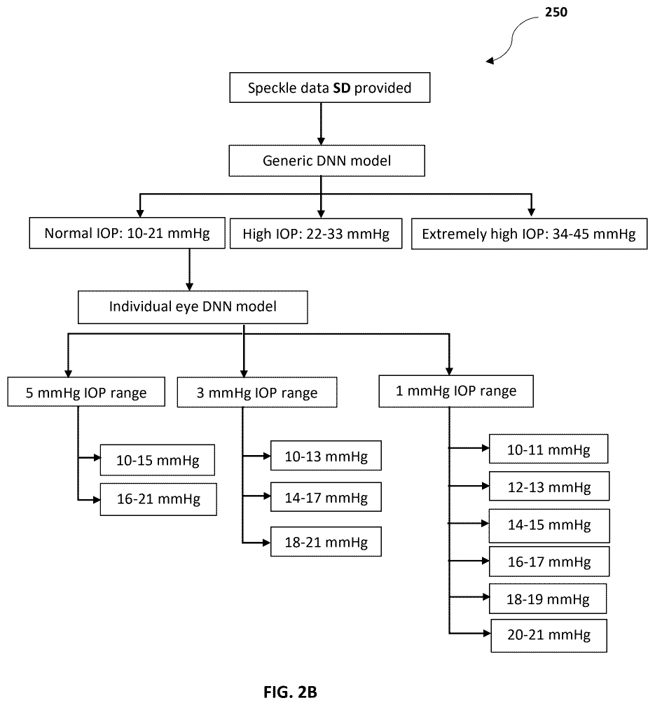

Reference is made to FIG. 2A illustrating, by way of a flow diagram 200, an exemplary method of the technique of the present disclosure. Measured data is provided (step 202) corresponding to video of M frames of speckle patterns, SP₁ . . . SPₘ (each of size N=p×q pixels) affected by the cyclic stimulation of external sound wave. This video constitutes the speckle data SD, which is transferred from the measured provider (e.g., measurement device 11) to the control system 22 or accessed by the control system 22 at the storage device, to be processed by the data processor 25.

The speckle data SD, i.e., the M frames of video, is preprocessed by the correlation module 30A by calculating 2D cross-correlations for every two consecutive frames (step 204). By this, a vector (of size M) containing cross correlation peaks of the recorded consecutive video frames is obtained (step 206).

Such vector representation of the speckle data undergoes DNN processing utilizing a predetermined generic DNN model (step 208), and the measured IOP range is extracted (step 210). The classification is performed in a discrete manner, requiring no calibration or prior knowledge about the tested eye. The goal of this stage is to identify abnormal IOP levels for further clinical examination.

In step 212 the measured IOP range is classified with respect to a normal IOP range (10-21 mmHg). If the measured IOP range is outside (typically above) the normal range, this is properly reported (via notification), as will be described below (step 214). If the measured IOP range is found to be within the normal range, the speckle data SD (its vector representation) is transferred to be further analyzed (by analyzer 32) to determine an individual IOP level/value (step 216), as will be described below. This analysis utilizes application of an individual DNN model to the vector representation of the speckle data SD (step 218) providing the individual's IOP level (step 220) with limiting achievable accuracy level of up to 1 mmHg.

Reference is made to FIG. 2B describing more specifically, by way of a flow diagram 250, an exemplary decision-making process performed at the IOP evaluation and IOP personalized measurement stages. The IOP classification is divided into two testing components. The first component is implemented by the generic DNN model-based processor 30 which operates to determine the IOP range in a discrete manner. As mentioned above, this stage does not require any calibration or prior knowledge about the tested eye.

The generic DNN model-based processor 30 subdivides the input data (vector representation of the speckle data) into one of three possible classes, each class with a range of 12 mm Hg. The classes are: 10-21 mmHg, 22-33 mmHg, and 34-45 mmHg representing the normal, high (i.e., abnormal), and extremely high IOP ranges, respectively. This classification permits rapid identification of both abnormal and normal ranges of the measured IOP range. Once an abnormal IOP range is identified, the classification is stopped, and corresponding notification is generated to direct the individual to further clinical investigation.

Once the measured IOP range is identified to be within the normal range, a second component, i.e., the individual DNN model based processor 32 operates to apply the individual DNN model to the speckle data (its vector representation) to determine the IOP level/value of each tested eye with an accuracy level of up to 1 mm Hg.

It is noted that this accurate analyzing step involves training a “personalized”/“individual” model for each specific individual using a train data set obtained by the technique of the present disclosure and corresponding data obtained using a known in the art IOP measurement technique, as was already described above.

A set of IOP sensitivity techniques (e.g., three such sensitivity techniques) is defined, where each sensitivity technique is characterized by a different sensitivity range of IOP values within the normal IOP range. As already described above, the generic DNN model, used for rapid identification of both abnormal and normal ranges of the measured IOP range, does not require calibration.

Once the authorized person (operator/physician) decides to further increase the accuracy of IOP measurement within the normal range (10-21 mmHg), a selected individual eye DNN model is to be used. To this end, the authorized person is required to perform a calibration stage, i.e., i) choose the specific sensitivity technique, i.e., sensitivity range, as will be described below, and (ii) create the respective DNN model for the individual eye (as described above). It should be noted that the individual specific DNN model may be created once for each individual and then used for further periodic medical IOP tests.

For example, a first sensitivity technique may utilize a binary classification task that classifies the measured IOP into two IOP ranges: 10-15 mm Hg and 16-21 mm Hg, each having a 6 mm Hg range. A second technique may be able to classify three different IOP ranges: 10-13 mm Hg, 14-17 mm Hg and 18-21 mm Hg. A third technique may be capable of classifying the exact measured IOP level with a deviation range of 1 mm Hg.

For the purpose of demonstrating the feasibility of the technique of the presently disclosed subject matter, pig eyes, which are remarkably similar to human eyes, were employed to provide a reliable data collection process with accurate ground truth, and to simulate clinically significant human glaucoma. Pig eyes are very similar to human eyes, having holangiotic (i.e., having blood vessels present in all parts of the retina) retinal vasculature, no tapetum, cone photoreceptors in the outer retina, and similar scleral thickness.

Measured data was collected from 24 pig eyeballs tested in a controlled environment. The laboratory was darkened and made silent to avoid background noises that could affect the installed system. The 24 tested pig eyeballs were acquired from a local distributor within less than two hours postmortem, and experiments were performed within eight hours following delivery. The eyeballs were fixed with 4% paraformaldehyde in 0.1 M phosphate buffer saline (PBS, pH 7.4) for four hours at 4 °C, after which the retinas were removed and flat-mounted with the retinal ganglion cell layer uppermost. They were then cover-slipped with PBS/glycerin (1:1).

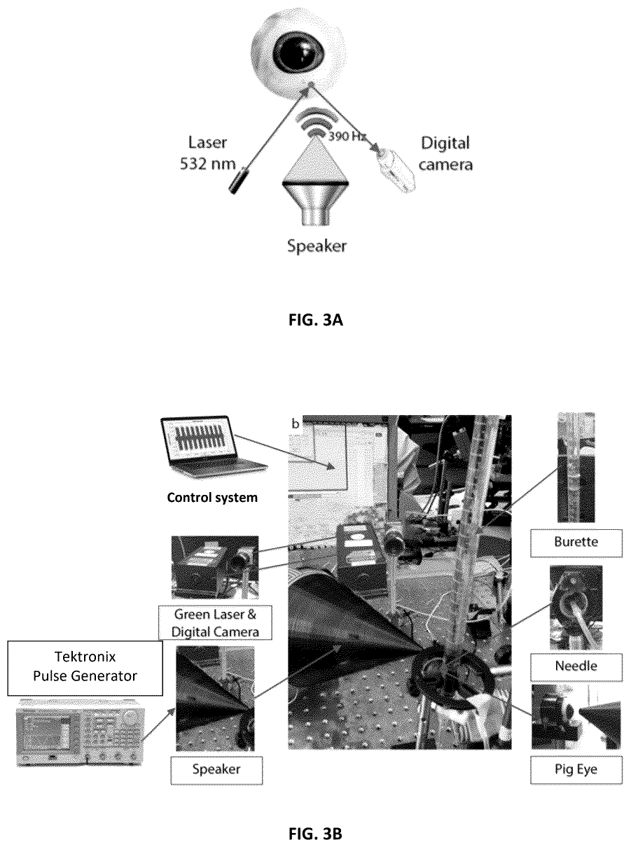

The measurement device (similar to the above-described device 11) was positioned at a distance of 35 cm from the tested pig eyeball. Since diffraction of the speckle occurs over a wide angle, no constraint exists concerning the position of the fast-imaging camera. A 532 nm green laser was positioned directly opposite the eyeball, as shown in FIG. 3A. The laser beam, covering a 3 mm diameter area, was fixed to be incident at a selected location of the sclera adjacent to the pupil. The 750 μW laser power was considered safe for human eyes. Speckle patterns reflected from the eye sclera were recorded using a Basler Aca1300-200 nm digital camera set for 1,000 frames per second (FPS), with a spatial resolution of 64×64 pixels with a pixel size of e.g., 5 microns. The camera's focal length was 55 mm, with F-number of 2.8. The camera was focused on the far field, defocusing the sclera and the speckle pattern. It is known (from the above-listed earlier patent publications of the inventor of the technique of the present application) that defocusing (slight defocusing) of the imaging optics (camera) with respect to an object's plane converts a tilting movement of the object into transversal movement of the speckles, i.e., causes the speckle pattern to move only in the transversal plane. For stimulation of the sclera, the inventors used a high-fidelity loudspeaker (Pioneer Ts-G1615R) with an excitation frequency of 390 Hz@105 dB, found to be highly responsive (after a sweep on frequencies between 130 and 1000 Hz).

Referring to FIG. 3B, an arbitrary waveform function generator (Tektronix, AFG3022B) controlled the speaker. For each recording, the sound wave agitated the pig eye by 26 consecutive cycles, each cycle comprising one second of stimulation followed by one second of a break. The frame rate of the digital camera was more than twice the stimulation frequency in order to meet the Nyquist ratio requirements. Each frame of the camera output contained a secondary speckle pattern. Using a MATLAB-based application, a computer (control system) receives the video files captured by the camera.

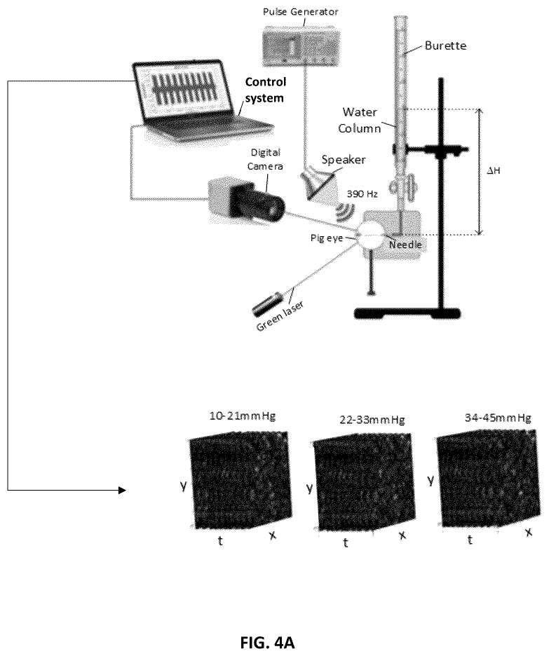

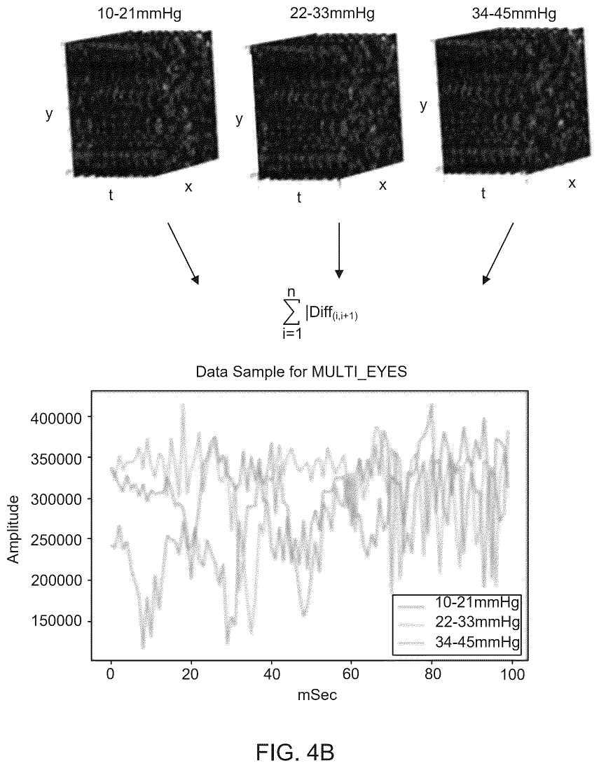

Reference is made to FIGS. 4A and 4B showing the IOP simulation and preprocessing according to the technique of the presently disclosed subject matter. The IOP of the pig eye sample was regulated by inserting a needle into the eyeball behind the eye (FIG. 4A). The needle was attached to a calibrated burette containing water by means of a flexible pipe to simulate IOP. While inserting the needle into the eyeball, the direction of insertion was controlled without validating the exact location inside the eye. Since the eyeball constitutes living tissue, it may be reasonably assumed that several needles experienced partial blockage, perhaps affecting the reference IOP. Such blockage could possibly explain the error rate, which increases between close IOP ranges since the sensitivity threshold of the system is limited.

The simulated eye pressure was measured in millimeters of mercury (mm Hg) and normal eye pressure was set in the range of 10-21 mm Hg. Each 1 mm Hg was considered equivalent to a 13.2 mm water column. Each eye was tested by a single needle penetration and the IOP was increased in steps of 1 mm Hg in the range of 10-45 mm Hg in order to obtain high accuracy in the normative pressure range. Above a pressure of 21 mm Hg, testing was performed with 2 mm Hg steps. The top IOP value was set at 45 mm Hg. Each tested eye was rejected after complete testing within one day under singular needle penetration.

It is important to note that all experiments were carried out in accordance with existing guidelines and regulations. Although deconstructed for laboratory optimization purposes, the device is entirely laser- and tissue-safe, as previously obtained from international regulators.

In order to classify the IOP, a continuous optical measurement session (video) while under sound stimulation is performed, where the measurement session contains multiple (generally two or more) successive stimulation cycles (pulsed mode of stimulation as described above) during continuous measurement. Each cycle/pulse includes an ON period of the increasing stimulation field (rise segment of the pulse) towards a STABLE stimulation followed by an OFF period of the decreasing stimulation value (fall segment of the pulse). Thus, during the continuous measurement session, the applied stimulation periodically increases and decreases, while the video of the reflected speckle patterns is acquired. FIG. 4A shows examples of the measurement sessions obtained for the different IOP ranges, where each measurement session includes a series of speckled images/frames (with two-dimensional respective (x, y) coordinates) obtained continuously over time, t.

In the experiments conducted by the inventors, a video of 52,000 frames was acquired, and this video was pre-processed for frame correlation extraction in order to classify the measured IOP range. For every two consecutive frames, the correlation was calculated using a fully discrete 2-dimensional linear cross-correlation with symmetrical boundary conditions, representing the shift between the two consecutive frames. This signal was normalized using the Manhattan norm as in Eq. (1) below.

$$norm = {\sum }_{i = 1}^{n} \vert {diff}_{( i , i + 1 )} \vert \qquad ( 1 )$$

where diff is the correlation between each two consecutive frames i, i+1, for each IOP sample/label (i.e., for each actual measured IOP setting) and n is the number of (pairs of) frames per single continuous stimulation session (e.g., 52,000 frames) belonging to the specific IOP sample/label.

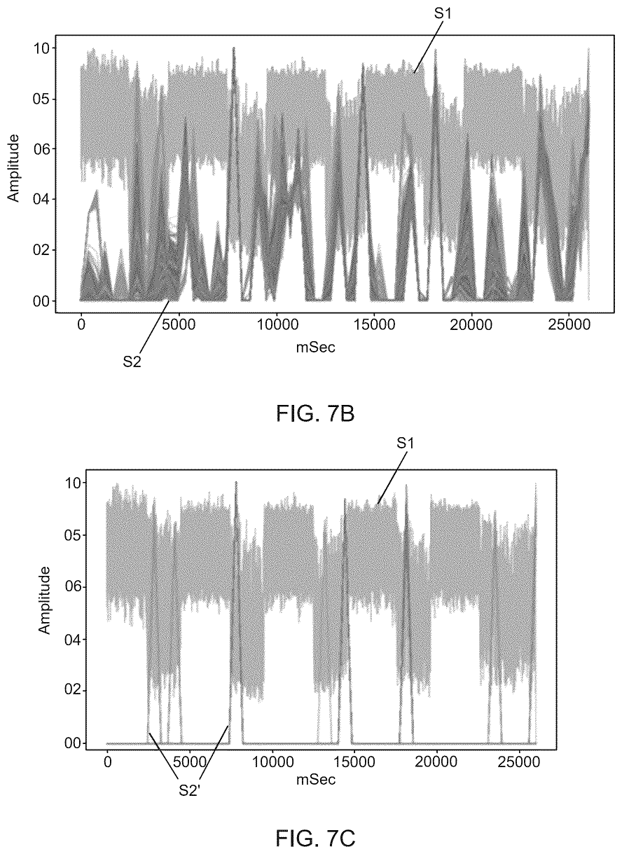

As part of the output, a one-dimensional array (signal S1 in FIGS. 7B and 7C) was created by pre-processing all the frames of each recorded video, constituting a full stimulation session belonging to a particular IOP sample/label. Quantitative assessment and comparison of our proposed method used the metrics shown in Eq. (3)-(6), where TP=True Positive; TN=True Negative; FP=False Positive; and FN=False Negative, calculated pixelwise by the logical operators given in Eq. (2).

$$\begin{matrix}{TP}_{i} = ( {x}_{i} == 1 ) \& ( {γ}_{i} == 1 ) {TN}_{i} = ( {x}_{i} == 0 ) \& ( {γ}_{i} == 0 ) \\ {FP}_{i} = ( {x}_{i} == 1 ) \& ( {γ}_{i} == 0 ) {FN}_{i} = ( {x}_{i} == 0 ) \& ( {γ}_{i} == 1 )\end{matrix} \qquad ( 2 )$$

$$accuracy = \frac{1}{n} {\sum }_{i = 1}^{n} \frac{{TP}_{i} + {TN}_{i}}{{TP}_{i} + {TN}_{i} + {FP}_{i} + {FN}_{i}} \qquad ( 3 )$$

$$precision = \frac{1}{n} {\sum }_{i = 1}^{n} \frac{{TP}_{i}}{{TP}_{i} + {FP}_{i}} \qquad ( 4 )$$

$$recall = \frac{1}{n} {\sum }_{i = 1}^{n} \frac{{TP}_{i}}{{TP}_{i} + {FN}_{i}} \qquad ( 5 )$$

$${F}_{1} = \frac{1}{n} {\sum }_{i = 1}^{n} \frac{2 {TP}_{i}}{2 {TP}_{i} + {FN}_{i} + {FP}_{i}} \qquad ( 6 )$$

where F₁ score is the harmonic mean of precision and recall and the tuple (xᵢ, yᵢ) represents, respectively, the model prediction (x) and the label (y) for sample (i.e., cross-correlated pair of frames) i. Here, x is the assignment/prediction of a sampled signal (one-dimensional array) based on the DNN model, and Y is its (ground truth) label.

Tests were conducted on separate dates upon receipt of the samples, and each pig eyeball was tested in one continuous session. The dataset contained roughly 20 million frames. Each pig eyeball video was given a unique identification consisting of the duration of measurement and the IOP reference value.

The videos from different recording days were subdivided into training and test datasets prior to subdivision into specific frames. Data for all tested eyeballs was included in the training and test sets, preventing any mixing between the two sets, which could have occurred with a simple random split.

As described above, the model input data for IOP classification is a vector containing cross-correlation peaks of the recorded consecutive video frames. This data was used to train a four-layer DNN model (generic DNN model) for IOP classification as will be described in detail below. The model was then applied separately to each of the three techniques for IOP classification.

IOP classification was divided into two testing components. The first component (generic classification) determined the measured IOP range in a discrete manner, requiring no calibration or prior knowledge about the tested eye. The main goal of this component was to identify abnormal IOP levels for further examination. The classification system subdivided the input signal into one of three possible classes with a range of 12 mm Hg. The first class was the normal range of 10-21 mm Hg. The second class, 22-33 mm Hg, represented the high IOP range. The third class, 34-45 mm Hg, represented the extremely high IOP range.

The second component determined the TOP level of each tested eye with an accuracy level of 1 mm Hg, requiring prior calibration. The process involves training a model for each individual by using one of the known in the art techniques for IOP measurement. To prove the feasibility of the method, the inventors focused on the normal IOP range of 10-21 mm Hg. A set of IOP sensitivity techniques was defined such that each successive technique improved accuracy over its predecessor. The first technique was a binary classification task that classified two IOP ranges: 10-15 mm Hg and 16-21 mm Hg, each having a 6 mm Hg range. The second technique was able to classify three different IOP ranges: 10-13 mm Hg, 14-17 mm Hg and 18-21 mm Hg. The latter technique was able to classify the exact measured IOP level with a deviation range of 1 mm. Each model was tested for each eyeball and compared with all tested eyeballs.

The generic and individual components of IOP monitoring allowed design of an IOP classification system that is accurate and general, permitting rapid identification of both abnormal and normal IOP.

The architecture of a non-limiting embodiment of the DNN-based model is schematically shown in FIG. 7A. The model output depended on the specific sensitivity technique. The first three layers within the DNN model were a combination of 1D convolution, batch normalization and rectified linear unit (ReLU), followed by a global average pooling operation. The last layer was a regular densely connected NN layer with a SoftMax activation function. The kernel size of each 1D convolutional layer was 3, with the corresponding number of filters being 64. The output of the network, representing the IOP classification resolution, depended on the specific IOP sensitivity technique.

The loss function was categorical cross entropy. During training, the loss was minimized using the Adam optimizer with β1=0.9, B2=0.999 and initial learning rate=0.001. Using the Reduce Learning-Rate on Plateau callback, the inventors reduced the learning rate when the validation loss stopped improving. This deep learning procedure was implemented with a batch size of 32 for 500 epochs on a single 1080Ti graphics processing unit (GPU) using a TensorFlow package.

As stated above, the generic remote IOP range classification method does not require any prior knowledge and the model predicts the IOP accurately. Table 1 shows the results of the generic and the individual components of IOP monitoring model classification.

TABLE 1

| | | | | |
|---|---|---|---|---|
| IOP range | Precision | Recall | F1 | Accuracy |

| Method | (mm Hg) | (%) | (%) | (%) | (%) |
|---|---|---|---|---|---|
| Generic photonic IOP | 10-21 | 98 | 97 | 97 | 91 |
| classification (without calibration | 22-33 | 81 | 84 | 82 | |
| or prior knowledge) | 34-45 | 72 | 70 | 71 | |

| | | | | | | |
|---|---|---|---|---|---|---|
| Individual | 5 mm Hg IOP | 10-15 | 85 | 74 | 79 | 80 |
| eye IOP | range | 16-21 | 77 | 87 | 81 | |
| classification | 3 mm Hg IOP | 10-13 | 87 | 82 | 84 | 83 |
| | range | 14-17 | 78 | 76 | 76 | |
| | | 18-21 | 85 | 91 | 87 | |
| | 1 mm Hg IOP | 10-11 | 82 | 79 | 80 | 70 |
| | range | 12-13 | 64 | 69 | 65 | |
| | | 14-15 | 66 | 58 | 60 | |
| | | 16-17 | 63 | 55 | 55 | |
| | | 18-19 | 69 | 76 | 70 | |
| | | 20-21 | 86 | 78 | 79 | |

The results of the DNN model validation indicate that the generic DNN model of the presently disclosed subject matter achieved an accuracy of 91% for classifying the measured IOP range into one of three IOP ranges: normal, high, and extremely high. The generic method maintains a high recall of 97% and high precision of 98% in the normal IOP range classification task.

The individual eye testing component is divided into three IOP sensitivity ranges—5 mm Hg, 3 mm Hg, and 1 mm Hg. The 5 mm Hg range attains an accuracy of 80% while maintaining a high recall of 87% for the 16-21 mm Hg range, and 85% precision for the 10-15 mm Hg range. The 3 mm Hg range attains an accuracy of 83% while maintaining a high precision of 87% for the 10-13 mm Hg range, and 91% recall for the 18-21 mm Hg range. The 1 mm Hg range attains an accuracy of 70% while maintaining a high precision of 82% for the 10-11 mm Hg range, and 86% precision for the 20-21 mm Hg range.

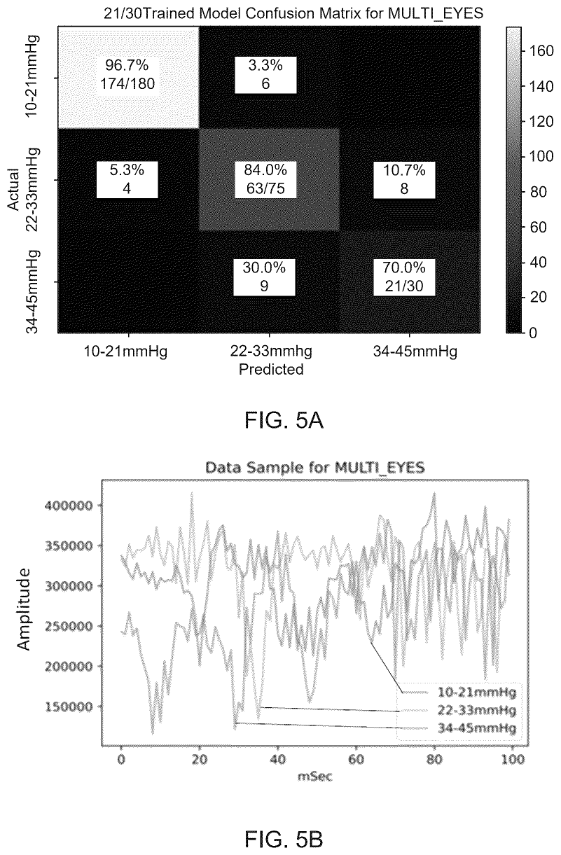

Reference is made to FIG. 5A which displays a confusion matrix of the trained generic model, and FIG. 5B which displays a 100-millisecond data sample of pre-processed pig eye speckle pattern displacement. The confusion matrix shows that the recall score of IOP detection of a single test sample (i.e., a single continuous stimulation session of a single tested pig eye) within the normal range is 97%, with a low error rate. The success rates for identifying high IOP ranges are also high and stand at 84% (22-33 mm Hg) and 70% (34-45 mm Hg), with almost all errors occurring between these two IOP ranges. The data plot presented in FIG. 5B shows a sample of the three IOP ranges classified by the generic model, each range marked by a different color. FIG. 5B shows that under the normal IOP range the amplitude variations are usually smaller than for the two high IOP ranges. The variance between the amplitudes for the different IOP ranges can be observed by eye even for a short time interval of 100 msec and indicates that inventors' definition of the IOP classification problem is accurate and that the suggested classification method of the presently disclosed subject matter is both sound and feasible.

It should be noted that AI approach allows for using spatial and temporal correlation data as well as wavelength-dependent data. The latter requires use of several lasers (in parallel).

The specific/individual analysis of the classification data permits more precise estimation of the IOP level at higher resolution. Using calibration and prior knowledge of the specific eye, this approach identifies each eye uniquely. The measurement procedures exemplified herein classify IOP sensitivity into three classes: 5-, 3- and 1-mm Hg. Table 1 shows the average of the three measurements metrics (Eqs. (3)-(5)) for all IOP ranges in the 24 tested eyes—these being precision, recall and accuracy.

The first analyzing stage of the classification data is a 5-mm Hg resolution step applied to two classes: 10-15 mm Hg, and 16-21 mm Hg. The accuracy of identifying TOP level for each range is about 80%. The second stage for the individual eye maintained a 3-mm Hg resolution step and shows an accuracy of 83%. Table 1 shows that the high values of the measurement metrics occur at the edges of the IOP ranges, i.e., 10-13 mm Hg, giving an F1 score of 84%, and 18-21 mm Hg, giving an F1 score of 87%, similar to the relative measurement metrics at the edges of the stage using 1-mm Hg resolution step: within the 10-11 mm Hg IOP range, model precision reached 82%, while for the 20-21 mm Hg IOP range it is 86%.

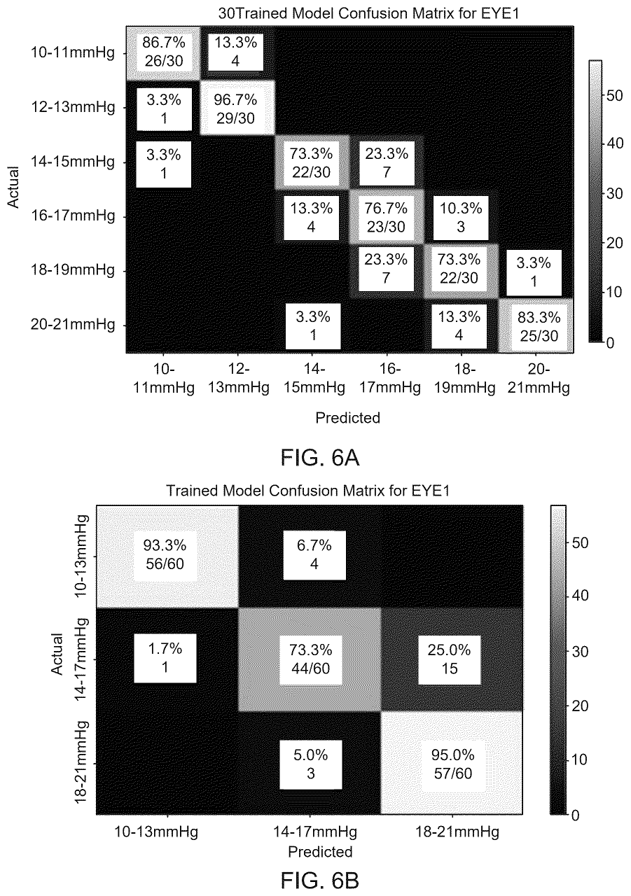

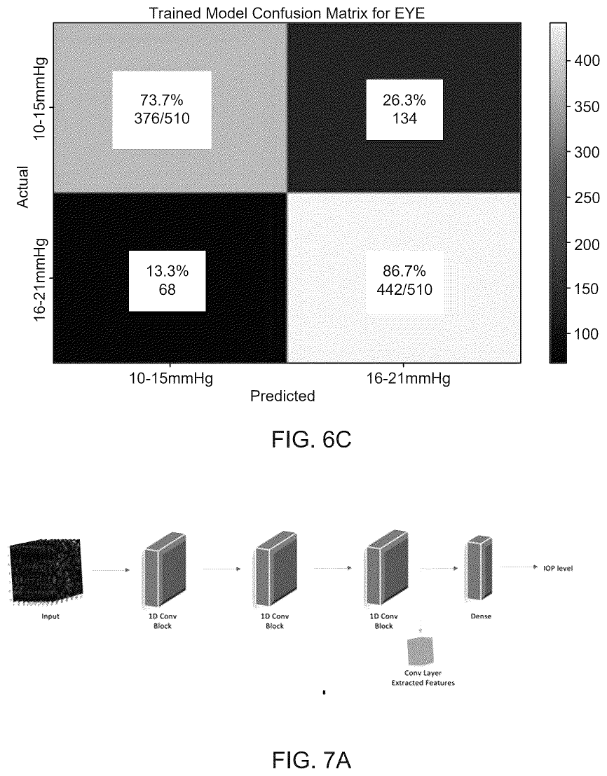

FIGS. 6A to 6C present the confusion matrices of the three mentioned stages of the analyzing of the classification data (measured IOP range) belonging to the individual IOP level extraction, showing the accuracy of the trained model on the test set. The results show that the trained individual DNN model errors are common for the near IOP ranges and are not dispersed across all possible IOP ranges, indicating the effective learning process of the model. In addition, the confusion matrix shown in FIG. 6A for the 1-mm Hg resolution step results variation shows that the high error rate occurs in the middle IOP ranges and not at the edges, as was noted above with reference to the results shown in Table 1. The values of these errors are also relatively low.

Reference is made to FIGS. 7A to 7C. FIG. 7A shows the DNN model architecture and FIGS. 7B and 7C show the feature extraction sample in the form of time variations of the filters used in the DNN model, where FIG. 7C shows the after “diff” variation (“diff” being the correlation between each two consecutive frames i, i+1). Also, FIGS. 7B and 7C show signal S1 (one-dimensional array being the result of cross-correlation between each two successive frames).

The DNN models were optimized during the training process. As was already described above, the DNN networks used in the presently disclosed subject matter are based on 1D convolutional layers, which are found useful for the IOP classification tasks due to their weight sharing, sparsity of connection capabilities, parameter efficiency, and other factors. Another main feature of the convolutional layers is the feature extractor, as can be seen in FIG. 7A (the features are extracted before the last densely connected NN layer). Extracting the trained convolutional layer filters by displaying them on a new input signal, as can be seen in FIG. 7B, allows to gain an understanding of the behavior of the model's decision-making process.

As shown in FIGS. 7B and 7C, the input signal had a duration of 25,000 milliseconds and was normalized in the range of (0, 1) to represent 5 consecutive cycles, each cycle comprising 2.5 sec of sound stimulation and 2.5 sec of break. Step-like/cyclic signal S1 represents the measurements, i.e., the vector of correlation data. In addition, FIG. 7B shows the multiple peak-shaped curves, generally S2, corresponding to the trained 64 convolutional layer filters representing the different filters used by the DNN model to classify the measured data and the final convolutional layer of the DNN. The corresponding amplitudes of the different filters express the confidence level of the decision, where 1 indicates the highest degree and 0 the lowest. Filters S2′ with amplitudes more than 0.8 were used after filtering layers as shown in FIG. 7C.

FIG. 7C demonstrates that the proposed DNN classifies the TOP level when the eyeball starts or stops reacting to the external stimulation signal, defined by the periodic vibratory profile.

It is noted that classification is achieved in every cycle of stimulation, either at the rise segment or fall segment of the stimulation pulse/cycle or during both of them, which stresses the importance of sampling and analyzing eye's response during the complete cycle of rising and falling stimulation.

Indeed, each IOP level involves variation of the eye weight, volume, and geometry, which in turn has a direct impact on the eye shape, direction, and speed of movement influenced by the agitating sound wave. Therefore, remote sensing micro-vibrations of the eye, induced by an external sound signal, provides evaluation/classification of the IOP level of the eye.

Thus, the inventors developed data analysis technique for processing and analyzing recorded measured data (e.g. one-dimensional vector representation thereof), corresponding to optical (contactless/remote) measurements of time variation of speckle patterns reflected from the eye sclera while being illuminated by coherent light and subjected to temporally encoded external acoustic stimulation, by utilizing DNN model based data processing to evaluate an IOP level of the eye.

The technique is completely contact-free, low-cost, and mobile. The system can be useful in early detection of glaucoma. The inventors have shown that eyes having high IOP range (higher than normal) can be accurately identified/classified.

As described above, the inventors succeeded in performing high IOP detection accuracy on 24 pig eyeballs using this hardware and software platform. The preliminary clinical feasibility of diagnosing high IOP, the cause of glaucoma, was demonstrated. The results of the generic DNN IOP model showed an accuracy of over 90% with near-perfect normative IOP detection.

1. A monitoring system for use in monitoring intraocular pressure, the system comprising a control system being configured as a computer system comprising data input and output utilities, memory and a data processor, wherein said data processor is configured and operable to process data indicative of measured data corresponding to sequence of acquisitions indicative of time variation of speckle patterns reflected from a region of interest in individual's eye while being subjected to coherent illumination and cyclic variation of predetermined acoustic stimulation and evaluate an intraocular pressure (IOP) level of the eye, said data processor comprising an IOP classifier configured and operable to apply a first predetermined model-based processing to said data indicative of the measured data and extract measured IOP range of the eye, and generate classification data indicative of the measured IOP range with respect to its relation to a normal IOP range.

2. The monitoring system of claim 1, wherein said data indicative of the measured data comprises one-dimensional vector representation of the measured data.

3. The monitoring system of claim 2, wherein said data processor further comprises a least one of the following:
a pre-processor configured and operable to generate the one-dimensional vector representation of the measured data; and
an IOP analyzer configured and operable to analyze the classification data indicative of the measured IOP range and, upon identifying that the measured IOP range is within the normal IOP range, selectively applying a second predetermined model-based processing to said one-dimensional vector representation of the measured data and extract an IOP level of the eye.

4. The monitoring system of claim 3, wherein said data processor comprises a correlation module configured and operable to calculate cross-correlations between every two consecutive speckle patterns in said sequence of acquisitions and create the one-dimensional vector representation of cross-correlation peaks.

5. (canceled)

6. The monitoring system of claim 2, wherein said first predetermined model-based processing comprises utilizing a predetermined generic model and classifying the measured IOP range, based on said one-dimensional vector representation, into one of three predefined classes of IOP ranges being normal, high, and extremely high IOP ranges.

7. The monitoring system of claim 6, characterized by at least one of the following:
said normal IOP range is 10-21 mm Hg;
said high IOP range is 22-33 mm Hg; and
said extremely high IOP range is 34-45 mm Hg.

8. (canceled)

9. (canceled)

10. The monitoring system of claim 2, wherein said data processor further comprises an IOP analyser configured and operable to analyze the classification data indicative of the measured IOP range and, upon identifying that the measured IOP range is within the normal IOP range, selectively applying a second predetermined model-based processing to said one-dimensional vector representation of the measured data and extract an IOP level of the eye, said second predetermined model-based processing utilizing at least one of the following,
individual models for determining the IOP value from the normal IOP range with increasing accuracy level;
an individual model for determining the IOP value with an accuracy level of 5 mm Hg in the normal IOP range; and
an individual model for determining the IOP value with an accuracy level of 1 mm Hg in the normal IOP range.

11. (canceled)

12. The monitoring system of claim 6, wherein said second predetermined model-based processing utilizes an individual model for determining the IOP value with an accuracy level of 3 mm Hg in the normal IOP range.

13. (canceled)

14. The monitoring system of claim 1, wherein said first model-based processing utilizes DNN models based on 1D convolutional layers.

15. The monitoring system of claim 14, wherein first three layers within the DNN model comprise a combination of 1D convolution, batch normalization and rectified linear unit, followed by a global average pooling operation.

16. The monitoring system of claim 1, wherein the control system is configured and operable for data communication with a measured data provider to obtain therefrom said data indicative of the measured data.

17. The monitoring system of receding claim 1, further comprising a measurement device configured and operable to provide the measured data, the measurement device comprising:
an optical unit configured and operable to perform one or more continuous imaging sessions, the optical unit comprising: a light source configured and operable to provide coherent illumination of at least one selected wavelength to illuminate a region of interest of an eye; and an optical detector detecting light reflected from the region of interest in the eye in response to said illumination and generating measured data for each of the at least one selected wavelength, said measured data comprising a sequence of acquisitions indicative of variation of speckle patterns in said reflected light;
a stimulation unit comprising an acoustic wave generator and applicator, the stimulation unit being configured and operable to perform one or more continuous stimulation sessions on said region of interest while under said imaging session, wherein each continuous stimulation session comprises a predetermined time pattern of stimulation cycles, thus applying to said region of interest a predetermined temporally encoded external acoustic waves, such that said variation of speckle patterns is affected by cyclic variation of the stimulation.

18. The monitoring system of claim 17, wherein said predetermined temporally encoded external acoustic waves comprise excitation frequency in a range of 100 to 500 Hz.

19. A monitoring system for monitoring intraocular pressure, the system comprising:
an optical unit configured and operable to perform one or more continuous imaging sessions, the optical unit comprising: a light source configured and operable to provide a coherent illumination of at least one selected wavelength to illuminate a region of interest of an eye; and an optical detector detecting light reflected from the region of interest in the eye in response to said illumination and generating measured data for each of the at least one selected wavelength, said measured data comprising a sequence of acquisitions indicative of variation of speckle patterns in said reflected light;
a stimulation unit comprising a sound wave generator and applicator, the stimulation unit being configured and operable to perform one or more continuous stimulation sessions on said region of interest while under said imaging session, wherein each continuous stimulation session comprises a predetermined time pattern of stimulation cycles, thus applying to said region of interest a predetermined temporally encoded external acoustic waves, such that said variation of speckle patterns is affected by cyclic variation of the stimulation;
a control system configured and operable to be responsive to the measured data to process said measured data and evaluate an IOP level of the eye, said processing comprising model-based processing.

20. The monitoring system of claim 19, wherein said predetermined temporally encoded external acoustic waves comprise excitation frequency in a range of 100 to 500 Hz.

21. The monitoring system of claim 19, wherein said control system comprises:
a processing utility configured and operable to perform a preprocessing of the measured data to obtain a one-dimensional vector representation thereof; and
an IOP classifier configured and operable to apply a first predetermined model-based processing to said one-dimensional vector representation of the measured data and extract measured IOP range of the eye, and generate classification data indicative of the measured IOP range with respect to its relation to a normal IOP range.

22. The monitoring system of claim 21, characterized by at least one of the following:
said control system further comprises an IOP analyzer configured and operable to analyze the classification data and IOP level and, upon identifying that the measured IOP range is within the normal IOP range, selectively applying a second predetermined model-based processing to said one-dimensional vector representation of the measured data and extract an IOP level of the eye,
said processing utility comprises a correlation module configured to calculate cross-correlations between every between every two consecutive speckle patterns in said sequence of acquisitions and create the one-dimensional vector representation of cross-correlation peaks; and
said first model-based processing comprises utilizing a predetermined generic model and classifying the measured IOP range, based on said one-dimensional vector, into one of three one of three predefined classes of IOP ranges being normal, high, and extremely high IOP ranges.

23. (canceled)

24. The monitoring system of claim 21, wherein said first predetermined model-based processing comprises utilizing a predetermined generic model and classifying the measured IOP range, based on said one-dimensional data vector, into one of three predefined classes of IOP ranges being normal, high, and extremely high IOP ranges being characterized by at least one of the following:
said normal IOP range is 10-21 mm Hg;
said high IOP 22-33 mm Hg; and
said extremely high IOP range is 34-45 mm Hg.

25. (canceled)

26. (canceled)

27. (canceled)

28. The monitoring system of claim 21, wherein said control system further comprises an IOP analyzer configured and operable to analyze the classification data and IOP level and, upon identifying that the measured IOP range is within the normal IOP range, selectively applying a second predetermined model-based processing to said one-dimensional vector representation of the measured data and extracting an IOP level of the eye, said second predetermined model-based processing being characterized by at least one of the following:
utilizing an individual model for determining the IOP value with an accuracy level of 5 mm Hg in the normal IOP range;
utilizes an individual model for determining the IOP value with an accuracy level of 3 mm Hg in the normal IOP range; and
utilizes an individual model for determining the IOP value with an accuracy level of 1 mm Hg in the normal IOP range.

29. (canceled)

30. (canceled)

31. (canceled)

32. The monitoring system according to claim 19, wherein said model-based processing utilizes DNN models based on 1D convolutional layers.

33. The monitoring system according to claim 32, wherein first three layers within the DNN model comprise a combination of 1D convolution, batch normalization and rectified linear unit, followed by a global average pooling operation.

34. The monitoring system according to claim 19, characterized by at least one of the following:
the stimulation unit comprises a loudspeaker; and
the detector unit is a camera.

35. (canceled)

36. A method for use in monitoring intraocular pressure, the method comprising:
providing data indicative of measured data corresponding to sequence of acquisition indicative of time variation of speckle patterns reflected from illuminated region of interest in individual's eye affected by cyclic variation of predetermined acoustic stimulation;
processing said data indicative of the measured data to evaluate an IOP level of the eye, said processing comprising apply a first predetermined model-based processing to said data and extracting measured IOP range of the eye, and generating classification data indicative of the measured IOP range with respect to its relation to a normal IOP range.

37. The monitoring system of claim 36, wherein said cyclic variation of predetermined acoustic stimulation comprises excitation frequency in a range of 100 to 500 Hz.

38. The method of claim 36, further comprising processing the measured data to obtain one-dimensional vector representation of the measured data, to be further processed by applying thereto the first predetermined model-based processing.

39. The method of claim 38, wherein said processing of the data indicative of the measured data further comprises analyzing the classification data indicative of the measured IOP range and, upon identifying that the measured IOP range is within the normal IOP range, selectively applying a second predetermined model-based processing to said one-dimensional vector representation of the measured data and extracting an IOP level of the eye.

40. The method of claim 39, wherein said classification data is indicative of predefined classes of IOP ranges being normal, high, and extremely high IOP ranges.

41. The method of claim 40, wherein said normal IOP range is 10-21 mm Hg.

42. The method of claim 39, wherein said high IOP range is 22-33 mm Hg.

43. The method of claim 39, wherein said extremely high IOP range is 34-45 mm Hg.
